(19) United States
(12) Patent Application Publication
(10) Pub. No.: US 2026/0265700 A1
(43) Pub. Date: Sep. 10, 2026

(54) NEURAL BIOHYBRID MANUFACTURED USING REDUCED GRAPHENE OXIDE NANOPARTICLES, NEUROMUSCULAR JUNCTION MODEL USING SAME, AND DRUG SCREENING METHOD USING SAME

(71) Applicant: Sogang University Research & Business Development Foundation, Seoul (KR)

(72) Inventors: Jeong-woo CHOI, Seoul (KR); Taehyeong HA, Seoul (KR); Minkyu SHIN, Paju-si (KR)

(73) Assignee: Sogang University Research & Business Development Foundation, Seoul (KR)

(21) Appl. No.: 19/657,852

(22) Filed: Apr. 24, 2026

Related U.S. Application Data
(63) Continuation of application No. PCT/KR2024/016510, filed on Oct. 28, 2024.

(30) Foreign Application Priority Data
Oct. 30, 2023 (KR) 10-2023-0146841

(51) Int. Cl. C12N 5/071; G01N 33/50

(52) U.S. Cl. CPC C12N 5/0697; G01N 33/5082; C12N 2513/00

(57) ABSTRACT

The present invention relates to a neural biohybrid, a neuromuscular junction model using same, and a method for screening a drug related to motor neuron diseases using same. The present inventors have experimentally verified that the neural biohybrid and the neuromuscular junction model using same of the present invention have an excellent effect on drug screening for treating amyotrophic lateral sclerosis (ALS), and thus the neural biohybrid and the neuromuscular junction model using same are expected to be applicable to drug screening and toxicity evaluation for various neurological disorders.

### CROSS-REFERENCE TO RELATED APPLICATIONS

This application is a continuation application of International Application No. PCT/KR2024/016510 filed on Oct. 28, 2024 which claims priority to Korean Patent Application No. 10-2023-0146841, filed on Oct. 30, 2023, the entire disclosures of which are incorporated herein by reference.

### TECHNICAL FIELD

The present invention was made with the support of the Ministry of Science and ICT under Project No. 1711180504 and Sub-Project No. 2019R1A2C3002300, and the research management institute for the project is the National Research Foundation of Korea, the research program is “Individual Basic Research Program (Ministry of Science and ICT)”, the project title is “Biohybrid Robot with Brain Assembloid-based Biomimetic Sensing Functions”, the lead institution is the Sogang University Research & Business Development Foundation, and the research period is from Jan. 1, 2023 to Feb. 29, 2024.

In addition, the present invention was made with the support of the Ministry of Science and ICT under Project No. 1711187608 and Sub-Project No. 2022M3H4A1A01005271, and the research management institute for the project is the National Research Foundation of Korea, the research program is “Nanomaterial Technology Development”, the project title is “Nano-Biohybrid Actuator Chip for Organoid-based Drug Screening”, the lead institution is the Sogang University Research & Business Development Foundation, and the research period is from Jan. 1, 2023 to Dec. 31, 2023.

In addition, the present invention was made with the support of the Ministry of Science and ICT under Project No. 1711198532 and Sub-Project No. RS-2023-00259341, and the research management institute for the project is the National Research Foundation of Korea, the research program is “Establishment of Collaboration Hubs for Leading Overseas Research Institutions”, the project title is “Sogang-UPenn Convergence Research Center for Theranostics of Emerging Infectious Disease”, the lead institution is the Sogang University Research & Business Development Foundation, and the research period is from Jul. 1, 2023 to Dec. 31, 2023.

In addition, the present invention was made with the support of the National Research Foundation of Korea (NRF) grant funded by the Korea government (MSIT) (RS-2024-00344633).

The present invention relates to a neural biohybrid, a neuromuscular junction model using same, and a method for screening a drug related to motor neuron diseases using same.

### BACKGROUND ART

The neuromuscular junction (NMJ) is the site where chemical and electrical signals are transmitted between motor neurons and muscle fibers in the human body, and plays a very important role in allowing muscles to contract smoothly. Motor neurons transmit signals to muscle fibers through the neuromuscular junction, resulting in muscle contraction. For muscle contraction to smoothly function in a neuromuscular junction model, a 3D structure with a skeletal muscle structure and motor innervation similar to that of humans is essential. In these skeletal muscle tissues, the integration of multiple motor nerves promotes the connection between muscle fibers and motor neurons and increase synaptogenesis at the neuromuscular junction.

Recently, carbon nanotubes, graphene and other nanomaterials have been used to create neuromuscular junction environments such as muscles and nerves due to their high electrical conductivity and biocompatibility. The development of such 3D in vitro models mimicking the human neuromuscular junction could provide a crucial tool for fundamental biological research, the development of regenerative medicine strategies, and drug screening for motor neuron diseases.

Unlike 2D cell culture, 3D spheroid-based systems promote the development of neural differentiation and maturation of human neural stem cells (hNSCs). Despite these advantages, current spheroid models often face necrotic issues due to the lack of microvasculature and limited oxygen diffusion during long-term in vitro culture. Consequently, insufficient cell-ECM interactions lead to inefficient neurogenesis and undesirable gliogenesis within the aggregated spheroids.

Currently, vascular cells within neurons directly support efficient distribution of oxygen and nutrients, and secrete factors such as glial-derived neurotrophic factor (GDNF), which promotes neuronal survival and axonal growth. In addition, graphene has garnered significant attention as one of the most promising biomaterials for biomedical applications due to its unique properties. Graphene-based materials can be used as scaffold materials to enhance neurogenesis, neural differentiation and the generation of neural stem cells (NSCs), offering significant potential for various applications.

Amyotrophic lateral sclerosis (ALS), also known as Lou Gehrig's disease, is one of the most fatal neurodegenerative diseases affecting motor neurons and is a rare disease with no clearly identified cause. As motor neurons, which transmit signals for movement from the brain to muscles gradually degenerate and are destroyed, body muscles become progressively weak, leading to a loss of the ability to control voluntary movements and premature death. To elucidate the mechanisms of these motor neuron diseases and develop drugs, research is underway to produce 3D neuromuscular junctions that function similarly to those in humans. These ALS-based neuromuscular junction models can provide a crucial tool for all fields of not only ALS research but also neurodegenerative and developmental neuroscience research.

### DISCLOSURE

### Technical Problem

The present inventors manufactured a

neuromuscular junction model comprising a biohybrid derived from an ALS patient and performed a comparative experiment treating it with an ALS drug, and confirmed that the neuromuscular junction model of the present invention has excellent effects in screening ALS drugs.

Accordingly, it is an object of the present invention to provide a neural biohybrid comprising carbon materials, vascular cells and neural stem cells.

It is another object of the present invention to provide a neuromuscular junction model manufactured by co-culturing: a neural biohybrid produced by culturing a mixture of carbon materials, vascular cells and neural stem cells; and a muscle bundle produced by culturing a hydrogel comprising muscle cells.

It is another object of the present invention to provide a method for producing a neural biohybrid, comprising the following step:
a culture step of culturing a mixture of carbon materials, vascular cells and neural stem cells.

It is another object of the present invention to provide a method for screening drugs related to motor neuron diseases, comprising the following steps:
a neural biohybrid production step of culturing a mixture of carbon materials, vascular cells and neural stem cells to produce a neural biohybrid; a muscle bundle production step of culturing a hydrogel comprising muscle cells to produce a muscle bundle; a neuromuscular junction formation step of co-culturing the muscle bundle and a hydrogel comprising the neural biohybrid to form a neuromuscular junction; a drug contact step of contacting a candidate substance with the muscle bundle; and a drug evaluation step of comparing the degree of muscle contraction in the muscle bundle contacted with the candidate substance to that in the muscle bundle not contacted with the candidate substance.

### Technical Solution

The present invention relates to a neuobiohybrid, a method for producing the same, a neuromuscular junction model using the same, and a method for screening drugs related to motor neuron diseases using the same.

The present inventors studied a method for producing neural biohybrids or brain organoids by integrating rGOp and HUVECs into hNSCs or IPSCs to mimic a neuromuscular junction similar to that of humans, and completed the present invention.

Hereinafter, the present invention will be described in more detail.

One aspect of the present invention relates to a neural biohybrid produced by culturing a mixture of carbon materials, vascular cells and neural stem cells.

In the present invention, the carbon materials may be one or more selected from the group consisting of reduced graphene oxide nanoparticles (rGOp), carbon nanotubes, carbon nanofibers and graphite, and for example, may be reduced graphene oxide nanoparticles, but are not limited thereto.

In the present invention, the vascular cells may be one or more selected from the group consisting of vascular endothelial cells (HUVECs), vascular endothelial progenitor cells and vascular smooth muscle cells, and for example, may be vascular endothelial cells, but are not limited thereto.

The rGO in the mixture may enhance neurogenesis and neural differentiation of the neural biohybrid.

The vascular cells embedded within the neural biohybrid may improve neuronal growth and neural network development through the efficient distribution of oxygen and nutrients.

One aspect relates to a neuromuscular junction model manufactured by co-culturing: a neural biohybrid produced by culturing a mixture of carbon materials, vascular cells and neural stem cells; and a muscle bundle produced by culturing a hydrogel comprising muscle cells.

In the present invention, the carbon materials may be one or more selected from the group consisting of reduced graphene oxide nanoparticles (rGOp), carbon nanotubes, carbon nanofibers and graphite, and for example, may be reduced graphene oxide nanoparticles, but are not limited thereto.

In the present invention, the vascular cells may be one or more selected from the group consisting of vascular endothelial cells, vascular endothelial progenitor cells and vascular smooth muscle cells, and for example, may be vascular endothelial cells, but are not limited thereto.

In the present invention, the muscle cells may be one or more selected from the group consisting of myoblasts, myocytes and myotubes, but are limited thereto.

In the present invention, the co-culture of the neural biohybrid and the muscle bundle may be performed for 3 to 28 days, preferably may be performed for 3 to 21 days, 3 to 14 days, 7 to 28 days, 7 to 21 days, and for example, may be performed for 7 to 14 days, but is not limited thereto.

Another aspect of the present invention is a method for producing a neural biohybrid, comprising the following step:
a culture step of culturing a mixture of carbon materials, vascular cells and neural stem cells.

In the present invention, the carbon materials may be one or more selected from the group consisting of reduced graphene oxide nanoparticles (rGOp), carbon nanotubes, carbon nanofibers and graphite, and for example, may be reduced graphene oxide nanoparticles, but are not limited thereto.

In the present invention, the vascular cells may be one or more selected from the group consisting of vascular endothelial cells, vascular endothelial progenitor cells and vascular smooth muscle cells, and for example, may be vascular endothelial cells, but are not limited thereto.

Another aspect of the present invention is a method for screening drugs related to motor neuron diseases, comprising the following steps:
a neural biohybrid production step of culturing a mixture of carbon materials, vascular cells and neural stem cells to produce a neural biohybrid; a muscle bundle production step of culturing a hydrogel comprising muscle cells to produce a muscle bundle; a neuromuscular junction formation step of co-culturing the muscle bundle and a hydrogel comprising the neural biohybrid to form a neuromuscular junction; a drug contact step of contacting a candidate substance with the muscle bundle; and a drug evaluation step of comparing the degree of muscle contraction in the muscle bundle contacted with the candidate substance to that in the muscle bundle not contacted with the candidate substance.

In the present invention, the carbon materials may be one or more selected from the group consisting of reduced graphene oxide nanoparticles (rGOp), carbon nanotubes, carbon nanofibers and graphite, and for example, may be reduced graphene oxide nanoparticles, but are not limited thereto.

In the present invention, the vascular cells may be one or more selected from the group consisting of vascular endothelial cells, vascular endothelial progenitor cells and vascular smooth muscle cells, and for example, may be vascular endothelial cells, but are not limited thereto.

In the present invention, the muscle cells may be one or more selected from the group consisting of myoblasts, myocytes and myotubes, but are limited thereto.

In the present invention, the co-culture of the neural biohybrid and the muscle bundle may be performed for 3 to 28 days, preferably may be performed for 3 to 21 days, 3 to 14 days, 7 to 28 days, 7 to 21 days, and for example, may be performed for 7 to 14 days, but is not limited thereto.

In the present invention, the motor neuron diseases may be one or more selected from the group consisting of amyotrophic lateral sclerosis (ALS), progressive bulbar palsy, pseudobulbar palsy, progressive muscular atrophy (PMA), progressive lateral sclerosis (PLS) and monomelic amyotrophy (MMA), but are not limited thereto.

### Advantageous Effects

The neural biohybrid produced in the present invention directly enhanced stem cell growth, neural network development, neurogenesis and neural differentiation, and when applied to the neuromuscular junction (NMJ) model, enhanced connectivity between NMJ motor nerve terminals and muscle fibers was observed compared to simple neural spheroids.

In addition, by comparing NMJs comprising ALS-biohybrids derived from ALS patients with normal biohybrids, it was confirmed that muscle contraction was reduced and the degree of muscle contraction was restored through treatment with the ALS drug bosutinib.

Therefore, it is expected that the neural biohybrid of the present invention may be applied to drug screening and toxicity evaluation for various neurological diseases.

### DESCRIPTION OF DRAWINGS

FIG. 1A is a schematic diagram showing a process for producing a muscle bundle according to one embodiment of the present invention.

FIG. 1B is a fluorescent image showing a muscle bundle produced according to one embodiment of the present invention, as confirmed by immunostaining.

FIG. 1C is a graph showing the degree of voluntary contraction of a muscle bundle produced according to one embodiment of the present invention.

FIG. 2A is a schematic diagram showing a process for producing normal and ALS neural biohybrids according to one embodiment of the present invention.

FIG. 2B is a fluorescent image showing a normal neural biohybrid produced according to one embodiment of the present invention, as confirmed by immunostaining.

FIG. 2C is a fluorescent image showing an ALS neural biohybrid produced according to one embodiment of the present invention, as confirmed by immunostaining.

FIG. 2D is a fluorescent image showing a neural biohybrid produced according to one embodiment of the present invention as confirmed by immunostaining using the HUVEC marker CD31, to confirm the presence of vascular endothelial cells (HUVECs) in the neural biohybrid.

FIG. 2E is a fluorescent image showing a comparison of the difference between an ALS neural biohybrid produced according to one embodiment of the present invention treated with bosutinib for 7 days and a control group not treated with bosutinib.

FIG. 3A is a schematic diagram showing a process for manufacturing a 3D neuromuscular junction model using a hydrogel comprising normal and ALS neural biohybrids according to one embodiment of the present invention.

FIG. 3B is a fluorescent image showing a 3D neuromuscular junction using a hydrogel comprising a biohybrid manufactured according to one embodiment of the present invention and a control spheroid, as confirmed by immunostaining.

FIG. 3C is a graph comparing the degree of recovery of muscle movement over time after treating a 3D ALS-neuromuscular junction produced according to one embodiment of the present invention with bosutinib.

FIG. 3D is a graph comparing the movement of muscle bundles to confirm the recovery effect of muscle movement at various concentrations of bosutinib according to one embodiment of the present invention.

FIG. 3E is a graph comparing the movement of muscle bundles to confirm the recovery effect of muscle movement at various concentrations of bosutinib according to one embodiment of the present invention.

### BEST MODE FOR CARRYING OUT THE INVENTION

The present invention relates to a neural biohybrid produced by culturing a mixture comprising a carbon material, vascular cells, and neural stem cells.

### Mode for Carrying Out the Invention

Hereinafter, the present invention will be described in detail by way of the following examples. However, these examples are only for illustrating the present invention, and the scope of the present invention is not limited to these examples.

### Example 1. Production of Muscle Bundles and Confirmation of Differentiated Muscle Bundles

3D muscle bundles mimicking human skeletal muscle structure using C2C12, which is one of the skeletal muscle cells, were produced.

First, a hydrogel used for the 3D muscle bundles was prepared using a mixture of 340 μl of C2C12, 30% Matrigel, 250 μl of 16 mg/ml fibronectin, 10 μl of thrombin (0.5 U/1 mg fibrinogen) and 100 μl of DMEM.

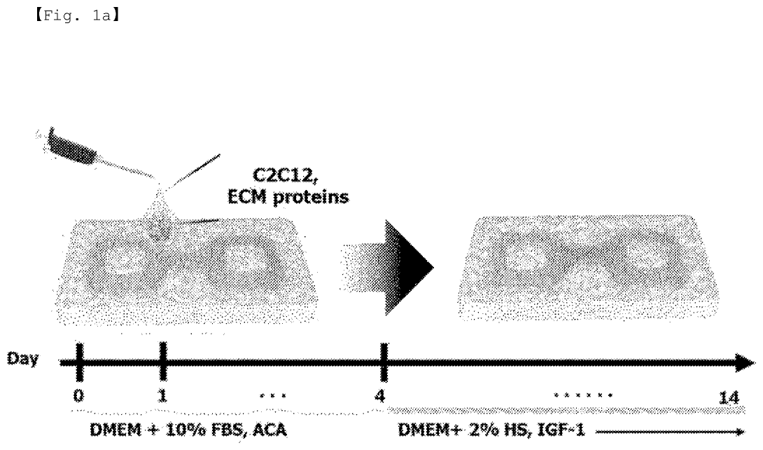

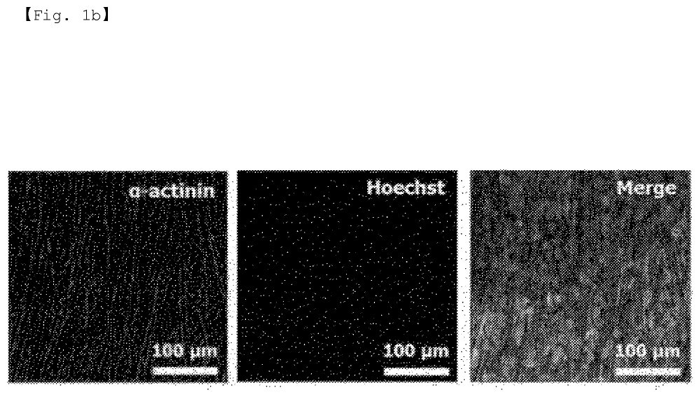

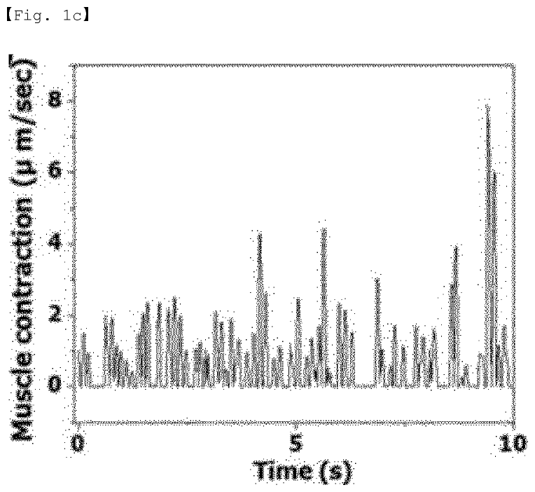

Subsequently, as shown in FIG. 1a, a hydrogel was cast into a pre-fabricated PDMS mold, and DMEM supplemented with 10% fetal bovine serum, 100 U/mL penicillin-G, 100 μg/mL streptomycin, and 1 mg/mL aminocaproic acid (ACA) was added as a muscle cell growth medium.

After culturing for 4 days, the medium was replaced with DMEM supplemented with 2% horse serum, 100 U/mL penicillin-G, 100 μg/mL streptomycin, 1 mg/mL aminocaproic acid, and 1 ng/mL insulin growth factor-1 (IGF-1) as a muscle cell differentiation medium. The medium was changed every 2 days, and differentiation was carried out for 14 days or more.

In order to confirm differentiation of muscle cells within the 3D muscle bundle, immunostaining was performed using α-actinin, a differentiation marker, and as shown in FIG. 1b, it was confirmed that muscle fibers were clearly formed within the muscle bundle. In addition, after differentiation for 14 days or more, it was confirmed that spontaneous muscle contraction occurred, as shown in FIG. 1c.

### Example 2. Production of Normal and ALS Neural Biohybrids and Confirmation of Differentiated Neural Biohybrids

To produce normal and ALS neural biohybrids, human neural stem cells (hNSCs) and neural stem cells derived from pluripotent stem cells induced from an ALS patient (ALS-hNSCs, a 55-year-old Caucasian female diagnosed with ALS) were grown on laminin-coated tissue culture plates with KnockOut™ DMEM/F-12 medium containing 20 ng/ml bFGF and 20 ng/ml EGF.

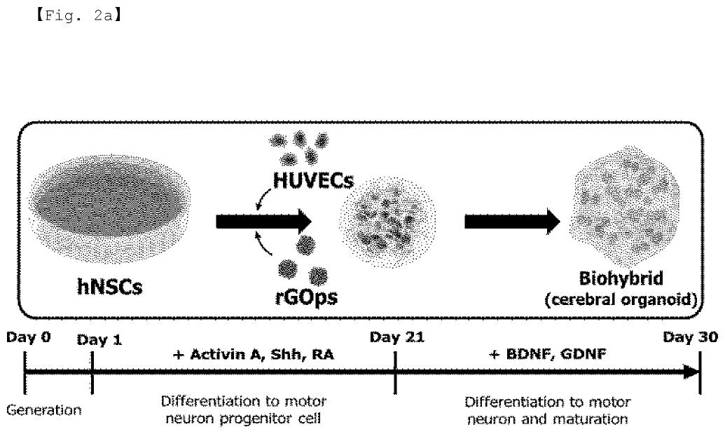

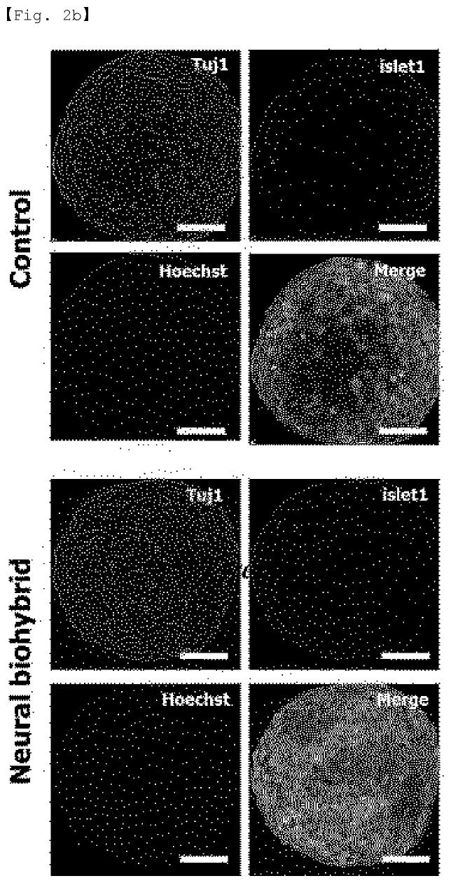

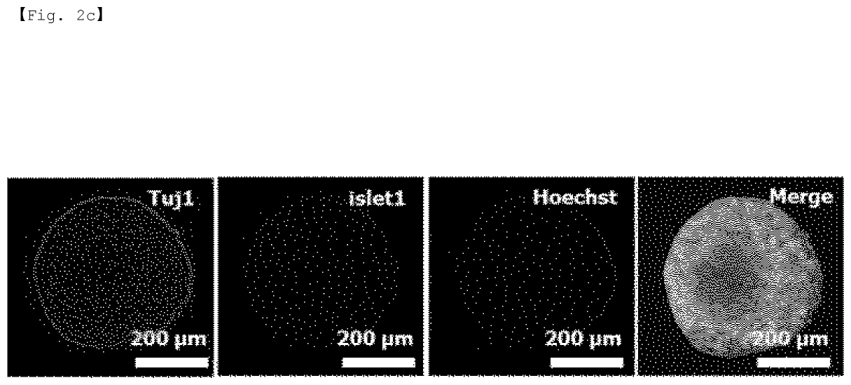

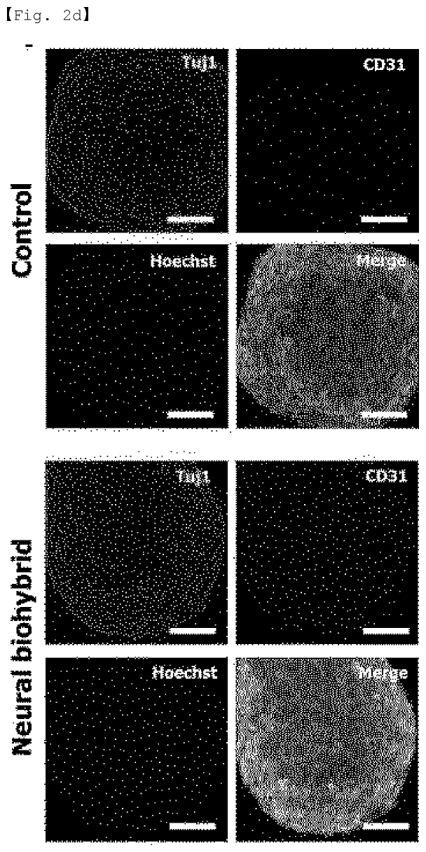

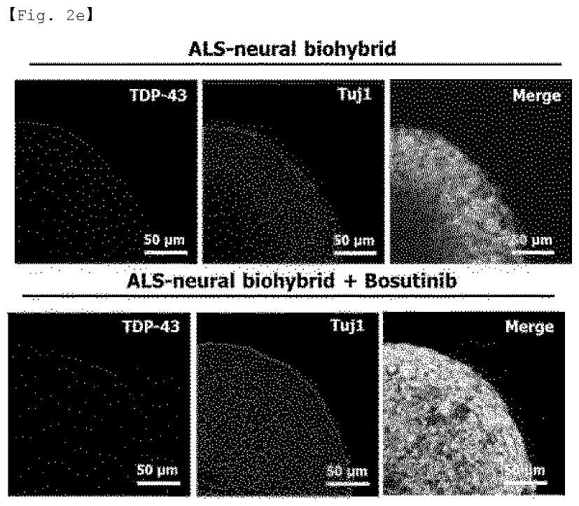

Neural spheroids were formed by mixing hNSCs and ALS-hNSCs (7.0×10⁴ cells per well), Human Umbilical Vein Endothelial Cells (HUVECs) (7.0×10³ cells per well), and 0.1 mg/mL reduced graphene oxide nanoparticles (rGOp), and seeding the mixture in a 96-well plate as shown in FIG. 2a. Forty-eight hours after cell seeding, the culture medium was replaced with a motor neuron differentiation medium containing 8 ng/mL bFGF, 200 ng/mL shh, 10 ng/mL activin A, and 50 μM retinoic acid. After 20 days of motor neuron differentiation, the medium was replaced with a motor neuron differentiation medium containing 10 ng/mL BDNF and 10 ng/mL GDNF for maturation of the neural biohybrids, and the cells were cultured for 8 days.

To confirm the differentiation of normal and ALS neural biohybrid, immunostaining was performed using the differentiation markers islet1 and Tuj1, and it was confirmed that the expression of motor neuron genes occurred significantly more than the conventional method, as shown in FIGS. 2B and 2C. In addition, it was confirmed that the vascular marker CD31 was expressed in the neural biohybrid comprising HUVECs as shown in FIG. 2D.

In addition, to confirm the ALS neural biohybrids, immunostaining was performed, confirming abnormal TDP-43 protein aggregation that was not found in normal neural biohybrids, and following treatment with the ALS drug bosutinib for 7 days, immunostaining confirmed a reduction in the amount of TDP-43 protein aggregation, as shown in FIG. 2E.

### Example 3. Production and Confirmation of 3D Neuromuscular Junctions Using Normal and ALS Neural Biohybrids

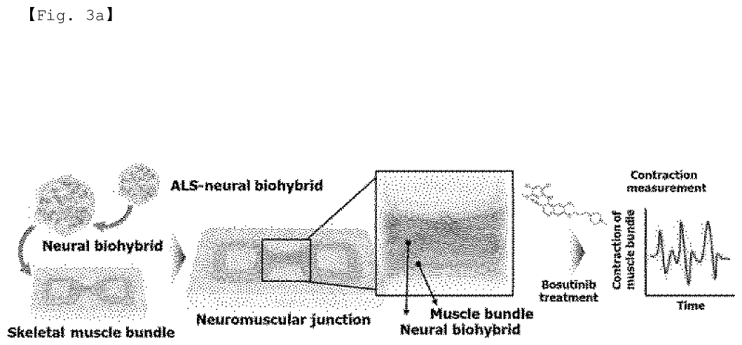

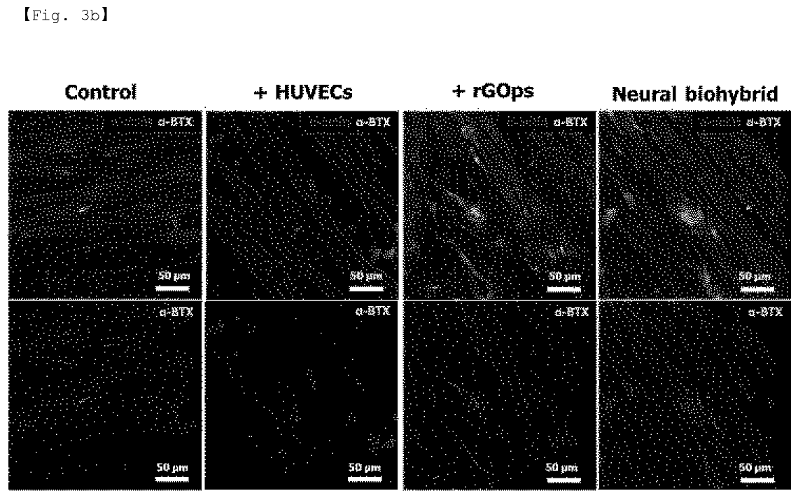

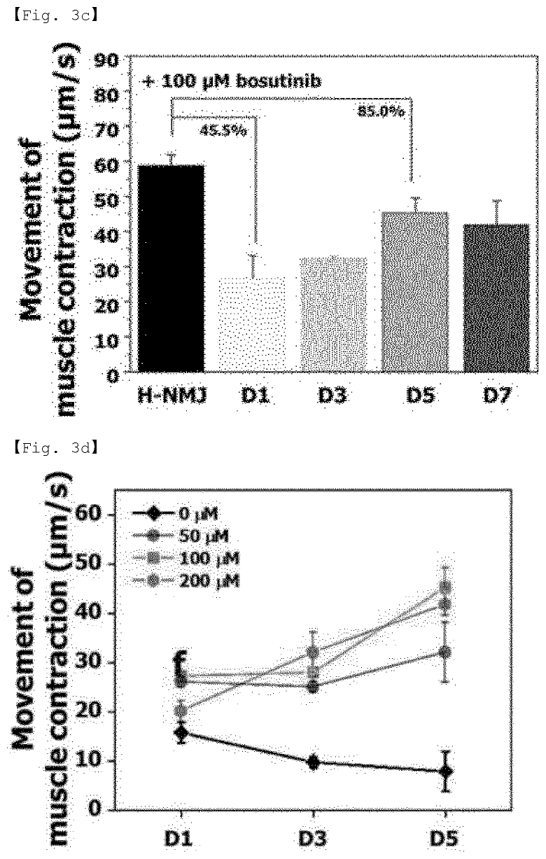

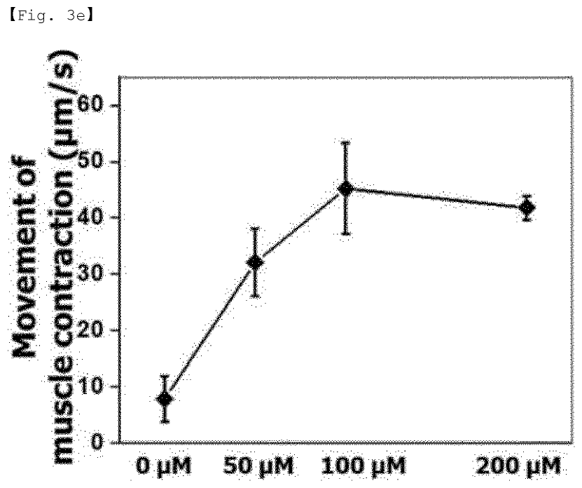

As shown in FIG. 3A, muscle bundles were produced through the formation and differentiation of muscle bundles in a PDMS mold, and then pre-differentiated normal and ALS neural biohybrids and CNT-COOH (0.1 mg/ml) and ECM proteins (30% Matrigel, 4 mg/ml fibronectin, 0.5 U/1 mg thrombin) were used to prepare a hydrogel, which was then further spread on top of the muscle bundles and co-cultured. The co-culture differentiation medium, along with muscle differentiation medium, was supplemented with brain-derived and glial cell line-derived neurotrophic factors (10 ng/ml BDNF and 10 ng/ml GDNF) to support viability of multiple neural biohybrids. The differentiation medium was replaced every two days for two weeks.

As shown in FIG. 3B, after 10 days of co-culture, NMJ formation was confirmed by immunostaining with the NMJ marker α-BTX, and it was confirmed that NMJs manufactured using neural biohybrids exhibited the highest level of α-BTX expression compared to conventional neural spheroids.

It was confirmed that NMJs manufactured using ALS neural biohybrids exhibited remarkably reduced muscle bundle movement compared to normal NMJs, and as shown in FIG. 3C, after 5 days of treatment with the drug bosutinib, it was confirmed that the contraction of the muscle bundle was restored to approximately 85% of the muscle movement of normal NMJs. In addition, muscle contraction was measured at various concentrations of Bosutinib, and it was confirmed that the greatest recovery of muscle movement was observed at 100 μM, as shown in FIGS. 3D and 3E.

### Industrial Availability

The present invention relates to a neuobiohybrid, a neuromuscular junction model using the same, and a method for screening drugs related to motor neuron diseases using the same.

1. A neural biohybrid produced by culturing a mixture of carbon materials, vascular cells and neural stem cells.

2. The neural biohybrid according to claim 1, wherein the carbon materials are one or more selected from the group consisting of reduced graphene oxide nanoparticles (rGOp), carbon nanotubes, carbon nanofibers and graphite.

3. The neural biohybrid according to claim 1, wherein the vascular cells are one or more selected from the group consisting of vascular endothelial cells (HUVECs), vascular endothelial progenitor cells and vascular smooth muscle cells.

4. A neuromuscular junction model manufactured by co-culturing:
a neural biohybrid produced by culturing a mixture of carbon materials, vascular cells and neural stem cells; and
a muscle bundle produced by culturing a hydrogel comprising muscle cells.

5. The neuromuscular junction model according to claim 4, wherein the carbon materials are one or more selected from the group consisting of reduced graphene oxide nanoparticles (rGOp), carbon nanotubes, carbon nanofibers and graphite.

6. The neuromuscular junction model according to claim 4, wherein the vascular cells are one or more selected from the group consisting of vascular endothelial cells (HUVECs), vascular endothelial progenitor cells and vascular smooth muscle cells.

7. The neuromuscular junction model according to claim 4, wherein the muscle cells are one or more selected from the group consisting of myoblasts, myocytes and myotubes.

8. A method for producing a neural biohybrid, comprising the following step:
a culture step of culturing a mixture of carbon materials, vascular cells and neural stem cells.

9. The method for producing a neural biohybrid according to claim 8, wherein the carbon materials are one or more selected from the group consisting of reduced graphene oxide nanoparticles (rGOp), carbon nanotubes, carbon nanofibers and graphite.

10. The method for producing a neural biohybrid according to claim 8, wherein the vascular cells are one or more selected from the group consisting of vascular endothelial cells (HUVECs), vascular endothelial progenitor cells and vascular smooth muscle cells.

11. A method for screening drugs related to motor neuron diseases, comprising the following steps:
a neural biohybrid production step of culturing a mixture of carbon materials, vascular cells and neural stem cells to produce a neural biohybrid;
a muscle bundle production step of culturing a hydrogel comprising muscle cells to produce a muscle bundle;
a neuromuscular junction formation step of co-culturing the muscle bundle and a hydrogel comprising the neural biohybrid to form a neuromuscular junction;
a drug contact step of contacting a candidate substance with the muscle bundle; and
a drug evaluation step of comparing the degree of muscle contraction in the muscle bundle contacted with the candidate substance to that in the muscle bundle not contacted with the candidate substance.

12. The method for screening drugs according to claim 11, wherein the carbon materials are one or more selected from the group consisting of reduced graphene oxide nanoparticles (rGOp), carbon nanotubes, carbon nanofibers and graphite.

13. The method for screening drugs according to claim 11, wherein the vascular cells are one or more selected from the group consisting of vascular endothelial cells (HUVECs), vascular endothelial progenitor cells and vascular smooth muscle cells.

14. The method for screening drugs according to claim 11, wherein the muscle cells are one or more selected from the group consisting of myoblasts, myocytes and myotubes.

15. The method for screening drugs according to claim 11, wherein the motor neuron diseases are one or more selected from the group consisting of amyotrophic lateral sclerosis (ALS), progressive bulbar palsy, pseudobulbar palsy, progressive muscular atrophy (PMA), progressive lateral sclerosis (PLS) and monomelic amyotrophy (MMA).
